(19) United States
(12) Patent Application Publication
(10) Pub. No.: US 2023/0329838 A1
(43) Pub. Date: Oct. 19, 2023

(54) METHODS FOR ORTHODONTIC TREATMENT PLANNING AND APPLIANCE FABRICATION

(71) Applicant: Ormco Corporation, Brea, CA (US)

(72) Inventors: Pavel Sergeevich Yablochkin, Moscow (RU); Karol Miranda, Tustin, CA (US); Anatoly Vasilevich Boltunov, Moscow (RU); Roman Alexandrovich Kolesnikov, Kolomna Town (RU)

(21) Appl. No.: 17/722,447

(22) Filed: Apr. 18, 2022

(51) Int. Cl. A61C 7/00; A61C 7/08; A61C 13/34

(52) U.S. Cl. CPC A61C 7/002; A61C 7/08; A61C 13/34

(57) ABSTRACT

A method for orthodontic treatment planning with aligners. The method includes determining an initial virtual pathway for each model tooth, calculating a space between adjacent pairs of model teeth at each stage, defining a minimum space between adjacent pairs of model teeth, and when a calculated space is less than the minimum space, creating a key-frame row in which each tooth position/orientation in the stage is a key frame. While a calculated space is less than the minimum space, iterating for each model tooth at the key-frame row, varying a position and/or orientation of the model tooth, and calculating a space between adjacent pairs of model teeth at each stage with the varied position and/or orientation of each model tooth at the key-frame row. A plurality of stages defines an orthodontic treatment plan. Each stage corresponds to an aligner for orthodontic treatment of a patient.

### TECHNICAL FIELD

The present invention relates generally to the field of orthodontic treatment and, more particularly, to systems and methods for orthodontic treatment planning.

### BACKGROUND

Orthodontics is the practice of manipulating teeth to correct malocclusions between the teeth of the upper and lower dental arches. Typically, treatment of malocclusions includes the use of an orthodontic appliance that applies corrective forces to the teeth. Over time, these corrective forces coerce the teeth to move into their orthodontically correct positions.

One way of applying corrective forces to teeth is an orthodontic appliance referred to as an “aligner.” Aligners are generally supplied as a series of removable appliances that incrementally reposition the patient's teeth from their initial orientation, in which the teeth may be maloccluded, to their orthodontically correct and aesthetic orientation. Patients being treated with aligners can insert and remove the aligners at will, and therefore do not need to visit the orthodontist to advance their treatment. Rather, when the currently worn aligner has moved the teeth to at or near a final orientation for that aligner, the patient merely begins using the next aligner in the series according to a treatment plan. In that regard, each aligner in the series differs from all other aligners in the series.

To fabricate aligners, the orthodontist first constructs a computer model of the patient's dentition. This model may be generated, for example, by taking an impression of the patient's dentition and then scanning the impression to digitize the impression for manipulation in a computer. Alternatively, the clinician may directly scan the patient's teeth with an intraoral scanner. The scanned data is then used to construct the computer model. In each case, the computer model includes one or more, preferably all teeth, in the patient's upper and/or lower jaws. Once the computer model has been constructed, the orthodontist may manipulate individual ones of the model teeth to ultimately determine a target orientation of each tooth that provides a corrected dentition for each respective jaw and which addresses any malocclusion and ideally provides an aesthetic smile. Multiple computer models may be generated prior to treatment. Each model may include a unique orientation of one or more model tooth in the dentition and may successively and incrementally reposition one or more model teeth from an initial orientation to a target orientation according to a treatment plan.

The incremental repositioning of the model teeth is then reproduced in a series of molds of the teeth. An aligner is formed from each mold. Where there are multiple molds, a set of aligners is manufactured with each aligner likely being unique to one of the molds. With each aligner, forces are imposed on the patient's teeth during orthodontic treatment. The patient's teeth may be moved incrementally from initial to target orientations according to the treatment plan as determined by the computer models. In this way, treatment moves the patient's teeth in a series of stages from an initial orientation that generally corresponds to the initial orientation of the computer model to a final orientation that generally corresponds to the target orientation of the computer model.

Manually planning the entirety of each stage is labor and time intensive and requires extensive training. For that reason, the planning processes may be automated. Yet, the uniqueness of each patient is a significant hurdle to full automation. One continuing challenge for automation is to reduce the time required to prepare a treatment plan that minimizes treatment duration for the patient while successfully treating the patient. Minimizing treatment duration reduces the costs because fewer stages of treatment reduce the material and modeling costs. However, at odds with minimization of time for treatment planning, during modeling, it is often necessary to manually manipulate a digital model of individual ones of the patient's teeth. That is, the automated, planned movements of one or more of the model teeth through their various treatment stages may need to be manually modified so that successful orthodontic treatment is more likely. In other words, automated tooth movement via software does not always result in a realistic orthodontic treatment plan.

For example, clinicians may need to manually resolve virtual tooth-to-tooth collisions that may occur as the model teeth are moved from their initial positions toward their final positions. Because tooth collisions can be identified during treatment planning and in the virtual environment, they can be resolved prior to actual treatment. Manually eliminating these collisions requires extensive training and practice and also consumes a technician's time.

While orthodontic treatment planning is generally successful, there remain problems in planning and in design prior to manufacturing of aligners according to a treatment plan. Thus, improved methods, systems, and computer program products are needed in orthodontic treatment planning and fabrication of aligners.

### SUMMARY

The present invention overcomes the shortcomings and drawbacks of methods and systems for modeling and manufacturing of aligners heretofore known for use in orthodontic treatment. While the invention will be described in connection with certain embodiments, it will be understood that the invention is not limited to those embodiments. On the contrary, the invention includes all alternatives, modifications and equivalents as may be included within the spirit and scope of the present invention.

In one aspect of the invention, there is a computer-implemented method for orthodontic treatment planning with one or more aligners. The method comprises determining an initial virtual pathway for each model tooth in the patient's jaw that is to be moved during orthodontic treatment. Each initial virtual pathway is from a first position and/or orientation of a model tooth in a first arrangement to a second position and/or orientation of the model tooth in a second arrangement. Each initial virtual pathway is divided into a plurality of stages with each stage of the plurality of stages defining a position and/or orientation of a respective one model tooth on the initial virtual pathway. The method further includes calculating a space between adjacent pairs of model teeth at each stage, defining a minimum space between adjacent pairs of model teeth permitted for orthodontic treatment, and when one or more of the calculated spaces is less than the minimum space, creating at least one key-frame row in which each tooth position and/or orientation in the stage is a key frame. While one or more of the calculated spaces is less than the minimum space, the method includes iterating one or more times of: for each model tooth at the at least one key-frame row, (i) varying the position and/or orientation of the model tooth from the first position and/or orientation or from a previous position and/or orientation from a previous iteration; and (ii) calculating a space between adjacent pairs of model teeth at each stage with the varied position and/or orientation of each model tooth at the at least one key-frame row. After iterating, the plurality of stages, in which each calculated space is greater than the minimum space, defines an orthodontic treatment plan in which each stage corresponds to an aligner for orthodontic treatment of a patient.

In one embodiment, the method further comprises manufacturing a series of aligners for the patient with each aligner corresponding to one stage of the plurality of stages.

In one embodiment, iterating includes varying a position of the at least one key-frame row in the plurality of stages.

In one embodiment, varying the position includes moving the at least one key-frame row by one stage earlier or one stage later in the plurality of stages.

In one embodiment, the at least one key-frame row includes a first key-frame row and a second key-frame row and wherein iterating one or more times includes varying the position and/or orientation of each model tooth at each of the first key-frame row and the second key-frame row.

In one embodiment, the first key-frame row and the second key-frame row are not consecutive stages.

In one embodiment, the minimum space is in a range of 0.0 mm to 0.04 mm.

In one embodiment, the method further comprises defining a maximum space, and wherein iterating occurs while one or more calculated spaces is less than the minimum space or is more than the maximum space.

In one embodiment, the position and/or orientation of each model tooth at each stage of the plurality of stages is defined in at least two degrees of freedom.

In one embodiment, the position and/or orientation of each model tooth is described as a vector with at least two components. In one embodiment, the vector is [symbol] =(α_{i,j}, β_{i,j})^{T} where X_{i,j} is the position and/or orientation of model tooth i at stage j in which i=1̅,̅ N̅ where N is a total number of model teeth in the patient's arch, j=1̅,̅ K̅ where K is related to a number of stages j of the plurality of stages, α is buccal/lingual motion of tooth i at stage j, and β is mesial/distal motion of tooth i at stage j.

In one embodiment, the at least one key-frame row includes a first key-frame row (Tₚ) and a second key-frame row (Tₚ₊₁) and the calculated space (Sᵢⱼ) between adjacent pairs of model teeth (i and i+1) at stage j is represented by: Sᵢⱼ=Fⱼ(\vec{X_{ι,p}}, \vec{X_{ι,p+1}}, \vec{X_{ι+1,p}}, \vec{X_{ι+1,p+1}}, Tₚ, Tₚ₊₁) in which calculated space (Sᵢⱼ) is a function Fⱼ at stage j of \vec{X_{ι,p}}, which is the position and/or orientation of model tooth i at the first key-frame row, \vec{X_{ι,p+1}}, which is the position and/or orientation of model tooth i at the second key-frame row, \vec{X_{ι+1,p}}, which is the position and/or orientation of model tooth i+1 at the first key-frame row, and \vec{X_{ι+1,p+1}}, which is the position and/or orientation of model tooth i+1 at the second key-frame row.

In one embodiment, stage j is between the first key-frame row (Tₚ) and the second key-frame row (Tₚ₊₁).

In one embodiment, defining the minimum space as zero and further including defining a maximum space between adjacent pairs of model teeth according to an inequality:

0<S_{i,j}<Sᵢ^{max} where Sᵢ^{max} is the maximum space between adjacent teeth i and i+1.

In one embodiment, the method further comprises defining a maximum movement velocity of each model tooth between any two consecutive stages. While one or more of the calculated spaces is less than the minimum space, the method includes calculating a movement velocity between each consecutive stage for each model tooth, and when the calculated movement velocity is greater than the maximum movement velocity, for each model tooth at the at least one key-frame row, varying the position and/or orientation of the model tooth from the first position and/or orientation or from a previous position and/or orientation from a previous iteration.

In one embodiment, the method further includes defining a deviation constraint for total movement of each model tooth from the first position and/or orientation. While one or more of the calculated spaces is less than the minimum space, the method includes calculating the total movement of each model tooth from the respective first position and/or orientation. When the calculated total movement is greater than the deviation constraint, for each model tooth at the at least one key-frame row, the method includes varying the position and/or orientation of the model tooth from the first position and/or orientation or from a previous position and/or orientation from a previous iteration.

In one aspect of the invention there is a computer-implemented method for orthodontic treatment planning with one or more aligners in which an initial staging plan includes a plurality of stages of treatment and indicates one or more collisions between adjacent pairs of model teeth. The method includes defining a minimum space between adjacent pairs of model teeth and creating at least one key-frame row in the initial staging plan. In the at least one key-frame row, each tooth position and/or orientation is a key frame. While at least one collision exists in the staging plan, the method includes iterating one or more times of: for each model tooth at the at least one key-frame row, (i) varying the position and/or orientation of the model tooth from an initial position and/or orientation or from a previous position and/or orientation from a previous iteration, and (ii) calculating a space between adjacent pairs of model teeth at each stage with the varied position and/or orientation of each model tooth at the at least one key-frame row. After iterating, the plurality of stages, in which each calculated space is greater than the minimum space, defines an orthodontic treatment plan in which each stage corresponds to an aligner for orthodontic treatment of a patient.

In one embodiment, the method includes manufacturing a series of aligners for the patient with each aligner corresponding to one stage of the plurality of stages.

In one embodiment, iterating includes moving the at least one key-frame row by one stage earlier or one stage later from a position in the initial staging plan.

In one embodiment, the at least one key-frame row includes a first key-frame row and a second key-frame row and iterating one or more times includes varying the position and/or orientation of each model tooth at each of the first key-frame row and the second key-frame row.

In one embodiment, the first key-frame row and the second key-frame row are not consecutive stages.

In one embodiment, the minimum space is in a range of 0.0 mm to 0.04 mm.

In one embodiment, the method further includes defining a maximum space, and wherein iterating occurs while one or more calculated spaces is less than the minimum space or is more than the maximum space.

In one embodiment, the position and/or orientation of each model tooth is described as a vector with at least two components.

### BRIEF DESCRIPTION OF THE DRAWINGS

The accompanying drawings, which are incorporated in and constitute a part of this specification, illustrate embodiments of the invention and together with the detailed description given below, serve to explain various aspects of the invention.

FIG. 1 is a flow chart of one embodiment of a method of orthodontic treatment planning for a patient.

FIG. 2 is a schematic view of a system according to one embodiment of the invention.

FIGS. 3A and 3B are 3-D digital tooth models of a patient's dentition with each tooth at an initial position prior to treatment, FIG. 3A depicts an upper arch and FIG. 3B depicts a lower arch.

FIGS. 4A and 4B are 3-D digital tooth models of a patient's dentition with each tooth at a final position following treatment, FIG. 4A depicts the upper arch corresponding to the 3D digital tooth model shown in FIG. 3A and FIG. 4B depicts the lower arch corresponding to the 3D digital tooth model of FIG. 3B.

FIG. 5 is a flow chart illustrating exemplary steps in a method for developing a treatment plan for a patient.

FIGS. 6A and 6B are charts showing stages according to an orthodontic treatment plan, FIG. 6A lists tooth contacts/spacing and FIG. 6B lists translational and rotational velocities associated with FIGS. 3A and 4A.

FIGS. 6C and 6D are charts showing stages according to an orthodontic treatment plan, FIG. 6C lists tooth contacts/spacing and FIG. 6D lists translational and rotational velocities associated with FIGS. 3B and 4B.

FIG. 7 is a flow chart illustrating exemplary method for reducing or eliminating interproximal collisions identified in the staging charts of FIGS. 6A and 6C.

FIGS. 8A and 8B are exemplary charts showing stages of tooth positioning according to an orthodontic treatment plan associated with the models shown in FIGS. 3A and 4A following elimination of tooth-to-tooth collisions shown for example in FIG. 6A.

FIGS. 8C and 8D are exemplary charts showing stages of tooth positioning according to an orthodontic treatment plan associated with the models shown in FIGS. 3B and 4B following elimination of tooth-to-tooth collisions shown for example in FIG. 6C.

FIG. 9 is a table of exemplary goals and constraints utilized in accordance with one embodiment shown in the exemplary flow chart of FIG. 7.

FIG. 10 is a perspective view of an aligner manufactured according to embodiments of the invention.

FIG. 11 is a schematic view of a computer device usable to determine staging according to embodiments of the invention.

### DETAILED DESCRIPTION

Embodiments of the invention are directed to computer-implemented methods, systems, and computer program products for orthodontic treatment planning, particularly for planning treatment of a patient's teeth with one or more dental aligners. Overall, the goal in treatment planning is to develop a plan to move the patient's teeth from their initial, pre-treatment positions (referred to as T1) to their final, post-treatment positions (referred to as T2). Embodiments of the invention are utilized to plan movement of one or more of the patient's teeth from one position to another position, such as any two positions from T1 to T2. Generally, these two positions may be any two positions of a single tooth at different times during planned movement of that tooth with an orthodontic appliance, such as an aligner. For example, the two tooth positions may include an initial tooth position and a second tooth position in consecutive stages of an orthodontic treatment plan.

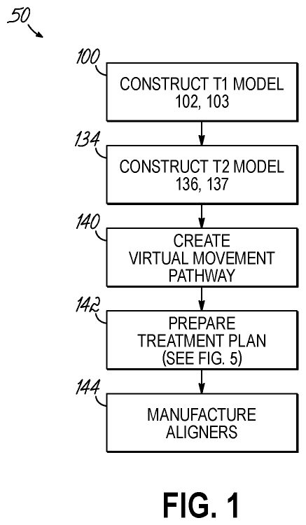

To that end and with reference to FIGS. 1-4B, a technician and/or clinician utilizes a virtual environment of a computer system within which to plan a patient's orthodontic treatment. According to embodiments of the invention, treatment planning involves virtual manipulation of a 3D digital model of the patient's teeth. As an example, treatment planning for utilization of aligners may include movement of one or more virtual teeth from their positions in a T1 model, which is a virtual model of the patient's teeth at T1, to their positions in a T2 model, which is a virtual model of the patient's teeth at T2. In this way, orthodontic treatment is first simulated with the aid of the computer system before aligners are manufactured to treat the patient.

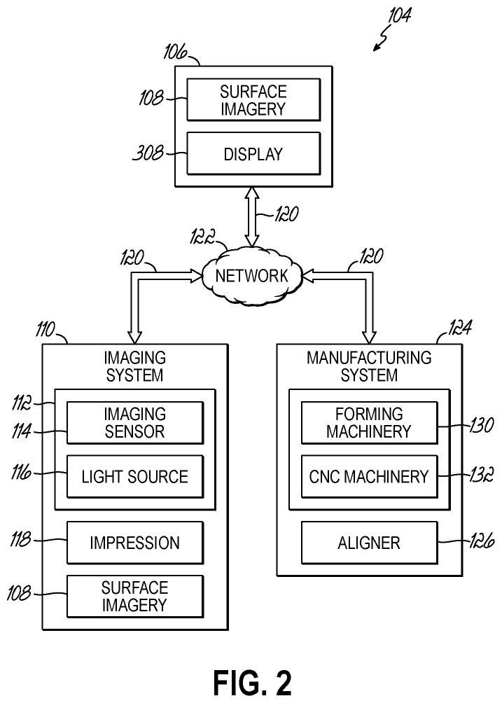

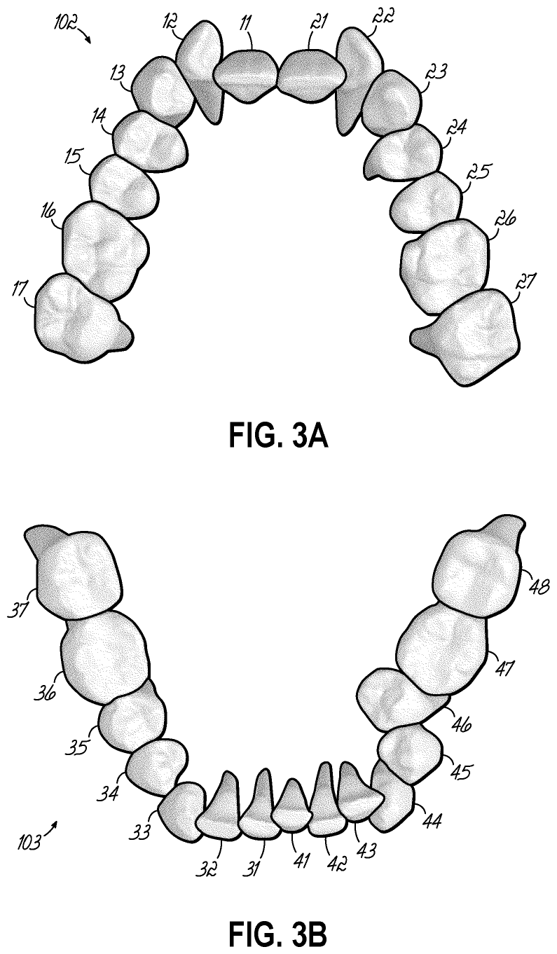

To plan virtual tooth movement according to one embodiment of a method of treatment planning 50 and with reference to FIG. 1, a 3-D digital model of a patient's dentition prior to orthodontic treatment is prepared at 100. This virtual model is referred to as a T1 model (exemplary T1 models are labeled 102, 103 in FIGS. 3A and 3B, respectively). An exemplary embodiment of a system 104 for preparing the T1 model 102, 103 is shown in FIG. 2. As shown, the system 104 includes a computer 106 and at least one imaging system 110. The computer 106 may receive multiple imageries 108 from imaging system 110 and/or another imaging system. The imaging system 110 may include a wand 112 having an imaging sensor 114 and a light source 116. The imaging sensor 114 may be an intra-oral camera that captures information regarding the oral cavity of the patient when exposed to light, for example from the light source 116.

The practitioner may insert at least a portion of the wand 112 into the patient's mouth. Using the light source 116 and the imaging sensor 114, the practitioner may capture data of all or selected crowns of the patient's teeth. Data may include surface imagery 108 of one or more of the patient's teeth or from an impression 118 of the patient's teeth and/or volumetric imagery (not shown) from volumetric data acquired from, for example, cone beam computed tomography (CBCT) or similar device.

The computer 106 may include any suitable computational device, such as a personal computer, a server computer, a minicomputer, a mainframe computer, a blade computer, a tablet computer, a touchscreen computing device, a telephonic device, a cell phone, a mobile computational device, dental equipment having a processor, etc. In certain embodiments, the computer 106 may provide web services or cloud computing services. More than one computer may also be used for storing data on a hard disk drive or flash drive and/or for performing the operations performed by the computer 106 in accordance with the embodiments of the invention. With surface imagery 108 and/or volumetric imagery, a 3-D digital model, i.e., the T1 model 102, 103 of the patient's teeth at T1 is produced at 100 in FIG. 1.

In the embodiment shown in FIG. 2, while not being limited to any particular network, the computer 106 is operatively coupled via one or more wired or wireless connections 120 to the imaging system 110 over a network 122. The network 122 may comprise a local area network, the Internet, an intranet, a storage area network, or any other suitable network. The computer 106 may use computer programs to model a patient's teeth, to design orthodontic treatment and appliances, and to model a patient's smile. The computer programs and systems have been proposed by applicant, examples of which are disclosed in U.S. Pat. No. 9,529,970 and International Application No. PCT/US2003/030917, filed on Sep. 26, 2003, and International Patent Application No. PCT/US2000/035558, filed Dec. 29, 2000, all of which are hereby expressly incorporated herein by reference in their entireties. These systems provide an interface through which a treating orthodontist or other dental practitioners can view, manipulate, communicate treatment, design preferences, and exchange patient data. Embodiments of the present invention provide enhancements to such systems that improve the functionality and utility of such systems and improve treatment efficiency while reducing treatment costs, as set forth herein.

While embodiments of the invention are not limited to the system 104 shown, the system 104 may also include or be in electrical communication with a manufacturing system 124 capable of manufacturing an aligner 126 (shown in FIG. 10). The manufacturing system 124 includes both forming machinery 130 and CNC machinery for machining 132 a trimline on each of the aligners 126, if necessary. By way of further example only, systems and methods of preparing 3-D models of a patient's teeth are known according to at least U.S. Pub. Nos. 2018/0165818 and 2014/0272772, which are assigned to Ormco Corporation and are incorporated by reference herein in their entireties.

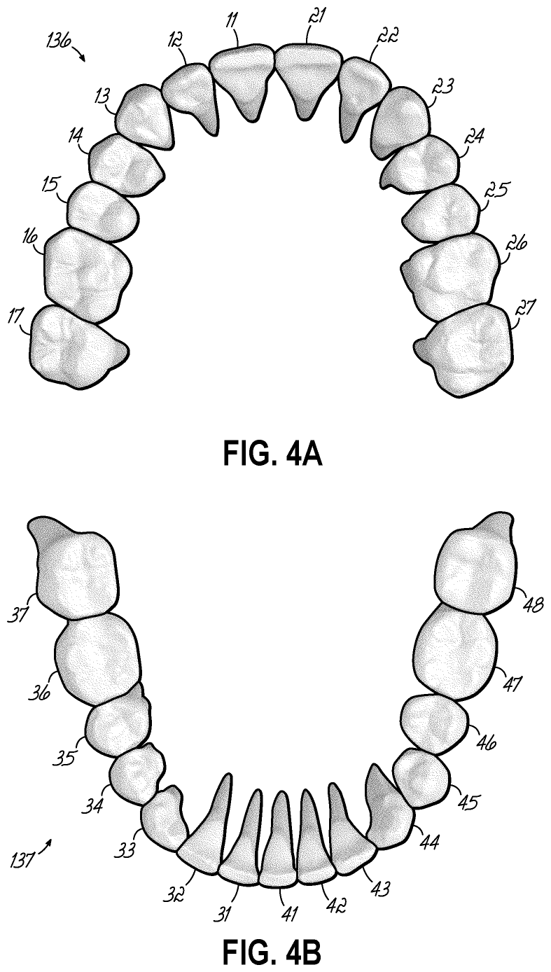

With further reference to FIG. 1, at 134, a digital orthodontic setup or a T2 model is then computed utilizing data in the T1 model. Exemplary T2 models corresponding to the T1 models 102 and 103 (FIGS. 3A and 3B) are labeled 136 and 137 and are shown in FIGS. 4A and 4B. Like the T1 model 102, 103, the T2 model 136, 137 is a virtual 3-D model. Whereas the T1 model 102, 103 represents a pre-treatment virtual arrangement of the patient's teeth, the T2 model 136, 137 represents an after-treatment arrangement of the virtual teeth shown in the T1 model 102, 103, minus any teeth that may be extracted prior to treatment. In other words, the T2 model 136, 137 is a virtual representation that corresponds to the desired outcome of orthodontic treatment. By way of example only and not limitation, the T2 model 136, 137 may be created by manipulating the image data 108 in the 3D Designer Software™ system running on the computer 106 (FIG. 2) available from Ormco Corporation, Orange, California.

Referring to FIG. 1 at 140, a program (such as the 3D Designer Software™ system) stored and operable on the computer 106 (FIG. 2) is configured to analyze the initial tooth positions for each tooth in the T1 model 102, 103 and the final positions for each tooth in the T2 model 136, 137. FIG. 3A illustrates an exemplary T1 model 102 for a patient's upper arch, and FIG. 3B illustrates an exemplary T1 model 103 for the patient's lower arch. The corresponding T2 models are shown in FIGS. 4A and 4B, respectively, in which FIG. 4A illustrates an exemplary T2 model 136 for the upper arch corresponding to the T1 model 102 for the upper arch shown in FIG. 3A, and FIG. 4B illustrates an exemplary T2 model 137 for the lower arch corresponding to the T1 model 103 for the lower arch shown in FIG. 3B. The program running on the computer 106 automatically creates a virtual pathway for movement of each tooth from its position in the T1 model 102, 130 (e.g., in FIGS. 3A and 3B) to its position in the T2 model 136, 137 (e.g., in FIGS. 4A and 4B, respectively). The calculation of the virtual pathway is not particularly limited to any technique. By way of example, the virtual pathway may be a shortest linear pathway between each tooth's position in the T1 model 102, 103 and the tooth's position in the T2 model 136, 137. By way of example only, a distance between a tooth at a T1 position in the T1 model 102, 130 to the tooth at a T2 position in the T2 model 136, 137 may be calculated based on the tooth movement velocity only. Movement velocity can be translational and/or rotational velocity. As an example, if the maximum permitted tooth movement velocity is 0.25 mm (i.e., translation) per stage and a distance between the T1 position and the T2 position is 1 mm, the virtual pathway between each stage will consist of a linear path having a length of 0.25 mm. Because this path may be refined as described herein, the virtual pathway calculated may be an initial calculation, that is, it is just a starting point for later calculations. As another example, the virtual pathway may be determined with each degree of freedom of each tooth's virtual motion being linearly interpolated from the T1 position to the T2 position. Therefore, for translational movement, if a coordinate for a model tooth at its T1 position is equal to 5.0 mm and the coordinate for the model tooth at its T2 position is equal to 6.0 mm, with linear interpolation as to that coordinate, the virtual pathway for the coordinate will be 5.1 mm, 5.2 mm, 5.9 mm, 6.0 mm for a total of 10 stages of movement. With the virtual pathway initially calculated, an orthodontic treatment plan is then prepared at 142. Once the orthodontic treatment plan is approved, typically by an orthodontist, aligners 126 are manufactured at 144, such as in the manufacturing system 124 of FIG. 2, for delivery to the patient.

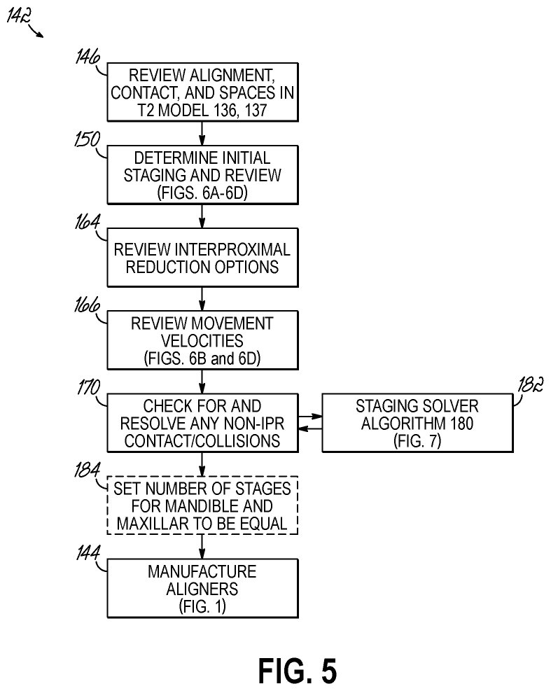

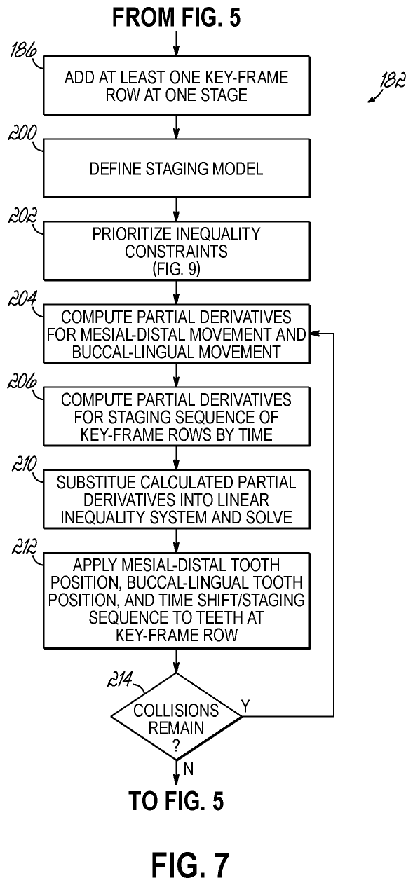

With reference to FIG. 5, an exemplary process for preparing an orthodontic treatment plan for one or both the T1 models 102 and 103, such as those shown in FIGS. 3A and 3B, according to 142 in FIG. 1 is shown. At 146 in FIG. 5, the technician may review each T2 model 136, 137 to make a final determination as to whether the final tooth alignment, the tooth-to-tooth contact (i.e., intra arch contacts between adjacent teeth in a jaw), and space between adjacent teeth are appropriate. The review may include ensuring that final contacts in the T2 model 136, 137 are 0 mm to 0.04 mm unless an interproximal reduction (IPR) is planned. With regard to the ranges, there may be some slight error in the locations of the teeth in the T1 model 102 and 103, which are reconstructed from scanned images of the patient's dentition. These errors may be on the order of 0.01 mm and are addressed by selecting the ranges, such as those provided herein. For example, while 0.04 mm in the range in this paragraph represents a slight overlap between adjacent model teeth, given the slight errors in scanning and reconstruction, 0.04 mm is selected as the lower limit of permitted overlap in the virtual model. Embodiments of the invention, however, are not limited by any range described herein.

At 150, in one embodiment, the software running on computer 106 may calculate initial staging for one or both the patient's dental arches based on the virtual pathway for each tooth from the T1 model 102, 103 to the T2 model 136, 137. Tooth movement along the virtual pathway is planned in stages. Staging is a process by which the sequence of tooth movements is determined in a treatment plan, and so staging is the timing, path, and order of the tooth movements. Each movement is planned from an initial tooth position (e.g., the T1 model 102, 103) to the final tooth position (e.g., the T2 model 136, 137). Where entirety of tooth movement is not possible or desirable in a single stage, multiple stages of movement are utilized. In other words, depending on the total amount of virtual movement required between the two positions for each tooth, the virtual movement pathway may be separated into one or more shorter movements. The shorter movement, when completed in separate stages, add up to the total movement required for treatment. For example, large movements (e.g., translational and/or rotational) may be sectioned into separate shorter translational and/or rotational movements that collectively equal the virtual movement pathway for each tooth from the T1 model 102, 103 to the T2 model 136, 137. Thus, from analyzing each tooth's virtual movement pathway, one or more stages of tooth movement is planned. Each stage is then associated with an aligner that is manufactured to move the patient's teeth according to the movement planned for that stage.

Staging determines successful orthodontic treatment but is often defined by physiological and material limitations. Exemplary physiological and material limitations include, but are not limited to, one or more of a maximum translation velocity, a maximum rotational velocity, and a maximum tip/torque velocity. These movement limitations may be predetermined for a particular patient, and by way of example, the maximum translation velocity may be 0.25 mm per stage, the maximum rotational velocity may be 2 degrees per stage, and the maximum tip/torque velocity may be 1 degree per stage. For each stage, the clinician or software may therefore limit virtual tooth movement according to one or a combination of each of a maximum of 0.25 mm of translational movement, a maximum of 2 degrees of rotational movement, and a maximum of 1 degree of tip/torque. These limitations may be associated with the patient's pain tolerance, with larger movements per stage being generally associated with greater pain, and/or being associated with the material limitations of the aligner. The planned staging may be shown in chart form and may be referred to as a staging plan.

In addition, the material from which the aligner is made may only permit a limited amount of movement over the time allotted for movement and so may set the maximum movement per stage if that is less than the patient's pain tolerance. The above limitations may be factors that determine the total number of stages required for treatment. As an example, the total number of stages may be determined by the tooth with the largest movement from its position in the T1 model 102, 103 to its position in the T2 model 136, 137. This tooth may be referred to as the lead tooth, and it may determine treatment length, because the tooth that moves the most dictates the overall number of stages based on a maximum permitted tooth velocity according to the physiological and material limitations. With these constraints, and possibly others described below, an initial staging chart may be calculated.

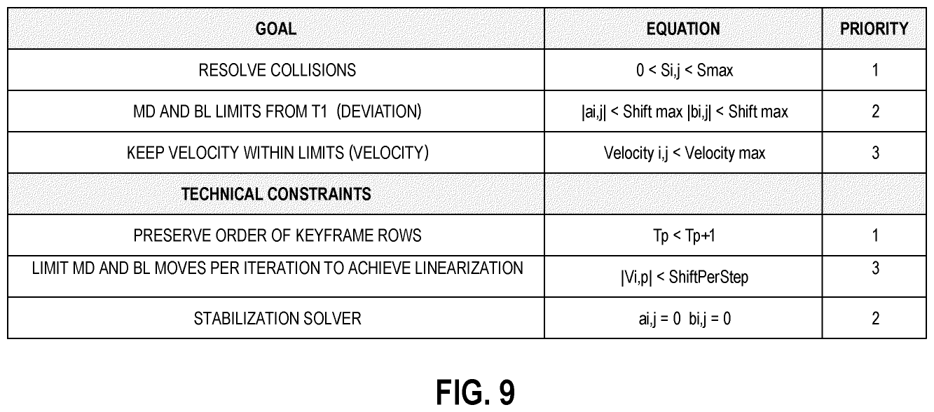

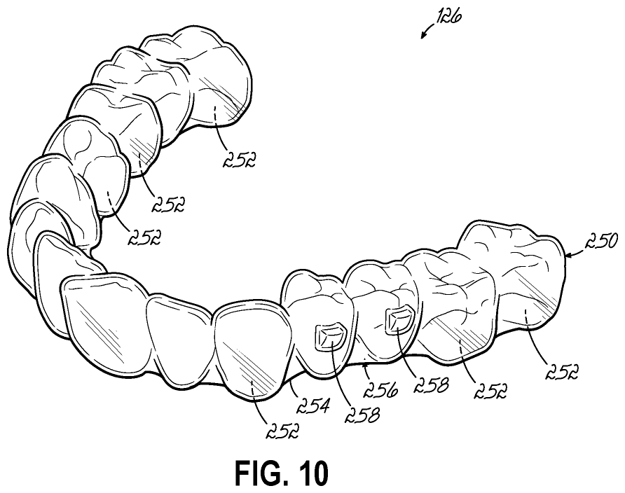

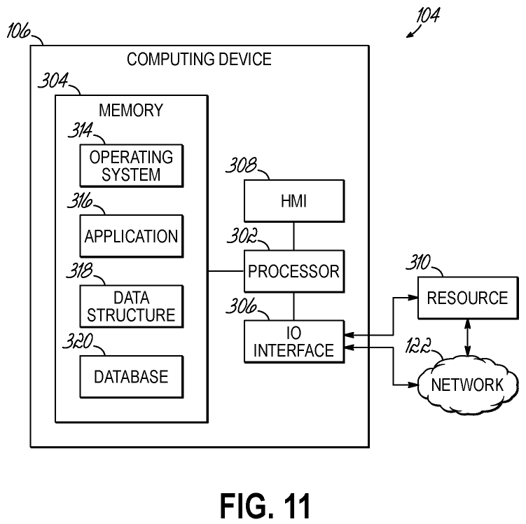

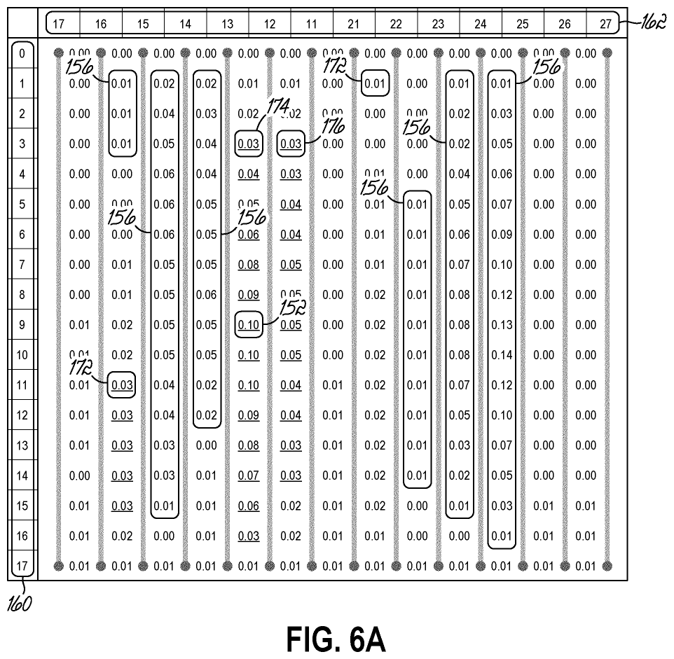

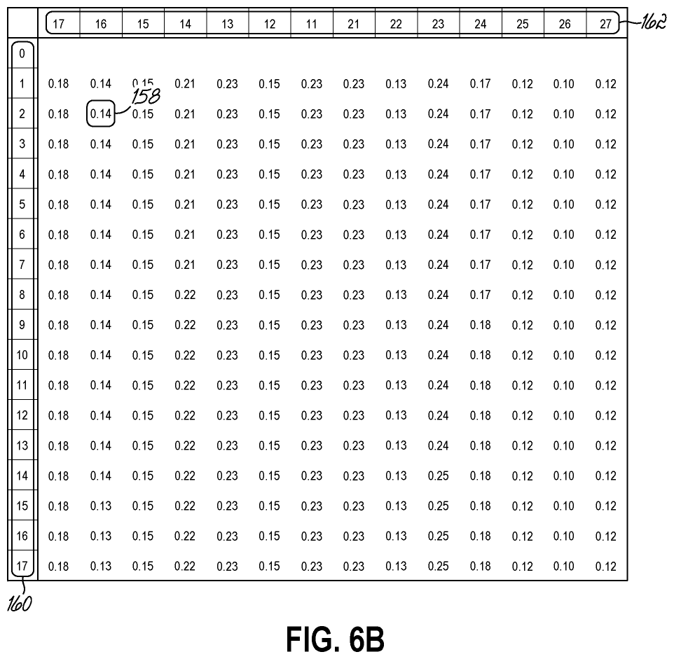

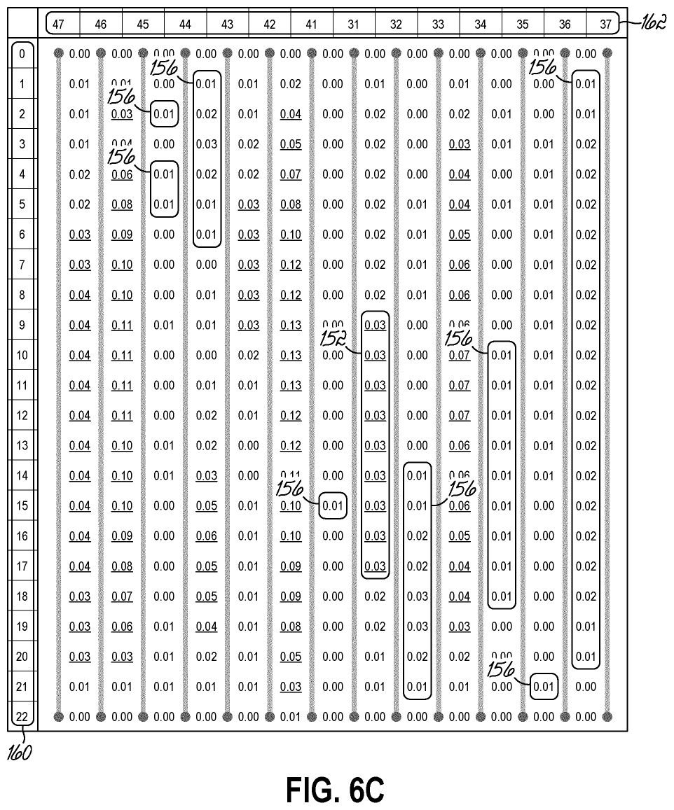

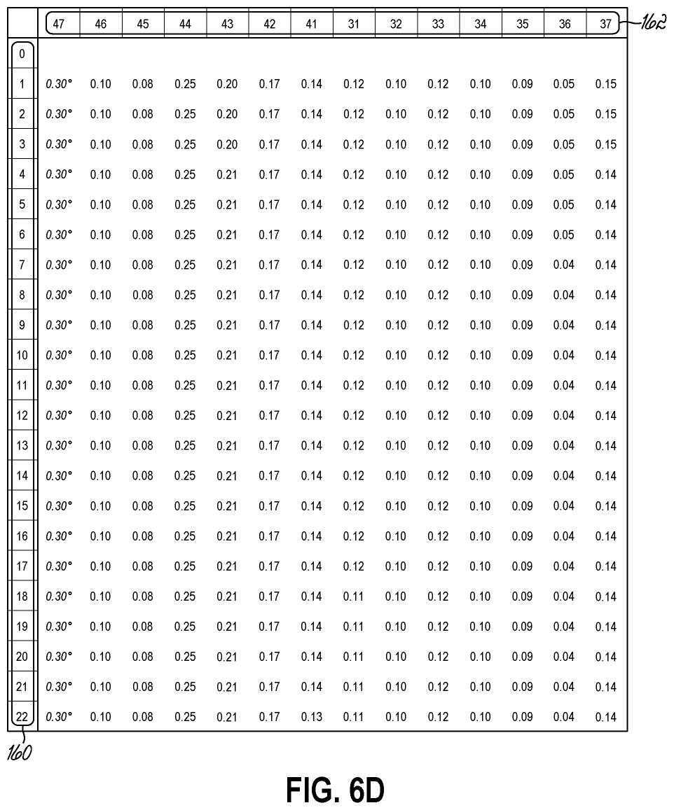

In view of the predetermined limitations and constraints described herein, initial staging charts at 150 in FIG. 5 to treat exemplary upper and lower arches are shown in FIGS. 6A and 6B based on the T1 models 102, 103 shown in FIGS. 3A and 3B and FIGS. 6C and 6D based on the T2 models 136, 137 of FIGS. 4A and 4B. That is, via the initial staging shown in FIGS. 6A (translational movement) and 6B (rotational movement), the T1 model 102 of FIG. 3A is transformed into the T2 model 136 shown in FIG. 4A. And, via the initial staging shown in FIGS. 6C (translational movement) and 6D (rotational movement), the T1 model 103 of FIG. 3B is transformed into the T2 model 137 shown in FIG. 4B.

In each of FIGS. 6A-6D, exemplary charts are shown with consecutive stages 160 listed down the left side of the chart with tooth numbers 162 from left to right at the top of the chart. Tooth numbers 162 are labeled according to Federation Dentaire Internationale (FDI) tooth numbering system and correspond to the tooth labels in FIGS. 3A-4B. With reference to FIGS. 6A and 6B, stage 0 represents the tooth positions shown in the corresponding T1 model 102, 103 (e.g., FIGS. 3A and 4A). The values in the chart of FIGS. 6A and 6C are the calculated space between teeth. The values may be positive (i.e., there is a gap between adjacent teeth) or negative indicated overlap between the virtual teeth. Stage 17 represents the tooth positions shown in the corresponding T2 model 136, 137 (e.g., FIGS. 3B and 4B). Thus, there are initially 17 stages of tooth movement planned to take the tooth arrangement of FIG. 3A to that of FIG. 4A, and there are 22 stages of tooth movement planned to take the tooth arrangement of FIG. 3B to that of FIG. 4B. Solid vertical lines with filled-in circles that extend from one stage and cross over one or more stages indicate tooth movement from the stage indicated and to the lower stage indicated. For example, in FIG. 6A, tooth 17 begins moving at stage 1 and moves during each stage until stage 17, where it reaches it's final position in the T2 model.

In the exemplary charts of FIGS. 6A and 6C, the calculated interproximal contacts or spaces between teeth are shown. For example, in FIG. 6A, the calculated contact between tooth 13 and tooth 12 at stage 9 is 0.10 mm of overlap (see circled value 152 in FIG. 6A). That is, according to the initial movement tooth 13 and tooth 12 are in collision at stage 9. In one embodiment, as shown, interproximal contacts in the T1 model (i.e., at the start of treatment corresponding to stage 0) may be adjusted to a value of 0, if the value at stage 0 indicates an overlap/collision with an adjacent tooth. In the T1 model, there shouldn't be any collisions or overlap because the T1 model is constructed from the patient's actual dentition. If the interproximal contacts at stage 0 indicate significant collisions, (e.g., more than 0.2 mm) then the T1 model may be revaluated for quality and, if necessary, additional imagery 108 may be collected to construct or reconstruct the T1 model according to 100 in FIG. 1. Other visual evaluations of the interproximal contacts for the T1 model at stage 0 may be made. By way of additional example only, where the patient's arches are considered “crowded” it makes sense that no spaces should be calculated between adjacent teeth. If a space is shown in the initial staging chart (e.g., FIGS. 6A and 6C) between adjacent teeth, the spacing shown in the chart can be visually confirmed with the T1 model as a preliminary quality evaluation of the T1 model relative to the initial staging calculation.

In the charts of FIGS. 6A and 6C, adjusted contacts are shown. By adjusted contacts it is meant that the value of the contact at stage 0 is subtracted/added to all stages. For that reason, at stage 0 all contacts are shown as “0.” Other information shown in the charts may include a visual indication of interproximal contacts or spaces between virtual teeth at each stage. In that regard, a range for various degrees of interproximal contact or space may be predetermined for ease of visual evaluation of a staging chart. For example, in one embodiment, normal contacts are those with contact values of 0.00 mm to 0.04 mm (this range includes a slight virtual overlap of adjacent teeth), which may be within the range of error for the imaging system 110. In FIG. 6A, normal contacts with values in a range of 0.00 mm to 0.04 mm, for example, are those which are not encircled or underlined. Normal contacts generally do not require modification and are acceptable in orthodontic treatment planning. While the identified ranges represent a slight overlap, as set forth above, embodiments of the invention are not limited to any preselected range, which may be related to error in the teeth surfaces from scanning and/or reconstruction of the scanned images. Further, a range may be selected in view of the desire to eliminate space between each pair of adjacent teeth in the patient's dentition during planned treatment. For that reason, a goal of treatment may be to have all interproximal spaces be zero in the T2 model.

With continued reference to FIGS. 6A and 6C, non-IPR contacts are contacts defined in a range of 0.05 mm to 0.14 mm. Non-IPR contacts are contacts that involve significant virtual overlap of adjacent virtual teeth. In FIG. 6A, for example, encircled area 152 (0.10 mm) is a non-IPR contact. In that regard, non-IPR contacts indicate significant interproximal tooth-to-tooth collisions during virtual tooth movement. That is, as a tooth is moved along its virtual movement pathway it may collide with another, adjacent tooth. Because the teeth are virtual 3D representations, they may overlap with one another. This overlap is indicated as a non-IPR contact and may be referred to as an interproximal collision in the sense that the adjacent virtual teeth move into contact with one another and thus are said to “collide” in virtual space. Non-IPR contacts, such as contact 152, are resolved prior to approval of an orthodontic treatment plan according to embodiments of the invention. Resolving non-IPR contacts is described below with reference to FIG. 5 at 170. Although not shown, non-IPR contacts may be color coded (e.g., red text) in the charts of FIGS. 6A and 6C for easy visual identification. Instead of color coding, all non-IPR contacts in FIGS. 6A and 6C are underlined for ease of comparison with later staging charts following collision avoidance calculations.

In FIG. 5, review of IPR contacts may occur at 164. IPR contacts may be those contacts greater than 0.14 mm, that is, a greater magnitude of overlap than non-IPR contacts. For example, IPR contacts may be defined in a range of 0.15 mm to 0.54 mm. No IPR contacts are shown in FIGS. 6A and 6C. These contacts are designated to be resolved by interproximal reduction at a predetermined stage of orthodontic treatment. IPR may be assigned to selected teeth with all IPR to occur during a single stage.

In addition, in FIGS. 6A and 6C, open space between teeth is shown at encircled areas 156. All open space is encircled and labeled 156. The distance between adjacent teeth may be displayed and differentiated from the normal contacts by colored text, such as green text or another visual textual indicator (e.g., plus sign). The staging charts of FIGS. 6A and 6C thus provide a visual, quantitative analysis of interproximal distances between adjacent teeth each stage of the initial staging.

Similarly, in the exemplary charts of FIGS. 6B and 6D, velocity of tooth movement (i.e., translational and rotational) between stages is displayed. For example, in FIG. 6B, the velocities of the tooth crown and/or tooth root may be displayed. The values shown in FIG. 6B represent the absolute movement (in mm) of each tooth along its virtual pathway between consecutive stages. For example, between stage 2 and stage 3, tooth number 16 is to move 0.14 mm (labeled 158). According to FIG. 6B, tooth number 13 and 23 are being moved at the greatest velocity of 0.23 mm to 0.25 mm and so likely determine the number of stages of treatment as described below. While in FIG. 6D, tooth number 47 is being rotated by 0.30 degrees per stage while tooth number 44 is being moved 0.25 mm per stage. Each of these teeth may be determining the number of stages of treatment in the orthodontic treatment plan.

With reference to FIG. 5 at 166, the clinician may review the movement velocities against predetermined minimum and/or maximum thresholds and evaluate whether the tooth movement is too slow/too fast under the given circumstances. This may include review of FIGS. 6B and 6D as described above. As is described herein, if the tooth movement meets or exceeds one or more of the predetermined limits, such as one or more of the maximum translational velocities, the maximum rotational velocity, and the maximum tip/torque velocity, described above, the clinician or the software may add one or more stages to reduce the per-stage movement velocity to below the predetermined limit.

In the exemplary embodiment, in FIG. 5 at 170, any non-IPR contacts, which may be referred to as intermediate, interproximal collisions herein, are addressed. With reference to FIG. 6A, for example, non-IPR contacts 172, 174, and 176 are indicated. These occur respectively in stages 11 (0.03 mm), 3 (0.03 mm), and 3 (0.03 mm), respectively, and must be resolved for successful orthodontic treatment. While the non-IPR contacts 172, 174, and 176 and others may be resolved by addressing each one manually by moving one or both the affected virtual teeth, according to embodiments of the invention, a staging solver algorithm 180 may be utilized to resolve all non-IPR contacts at 182. Advantageously, the staging solver algorithm 182 reduces or eliminates manual repositioning and consequently reduces the clinician's involvement with orthodontic treatment planning.

Once any collisions are resolved according to 182, in one embodiment, treatment planning according to 142 in FIG. 5 may include predetermined constraints on the total number of stages. For example, at 184, the number of stages for treatment of the patient's upper arch and the number of stages for treatment of the patient's lower arch may be made the same by default. The arch that requires the greatest number of stages (e.g., the arch with the lead tooth) determines the number of stages for the other arch. For example, as between FIGS. 6A and 6C, the lower arch (FIG. 3B) requires 22 stages, whereas the patient's upper arch requires 17 stages to move all the patient's teeth in their upper arch from their T1 positions to their T2 positions. In one embodiment, at 184, the treatment plan for the patient's upper arch is lengthened to 22 stages to match the number of stages required to treat the lower arch. However, the number of stages is not limited to a minimum number or a maximum number. For reference, the number of aligners 126 planned for treatment is 22 per arch (i.e., a total of 44 aligners), which may then be manufactured at 144 according to FIG. 1. The number of aligners 126 thus corresponds to the number of stages for treatment of the respective arch.

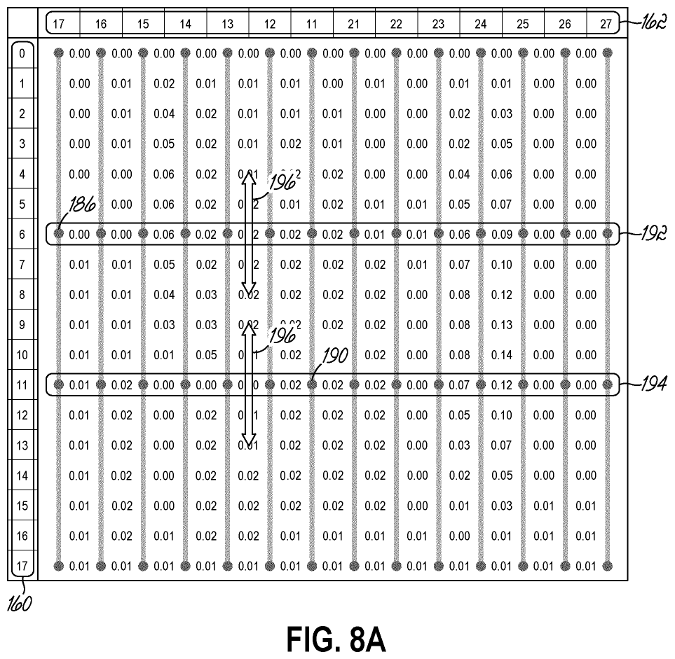

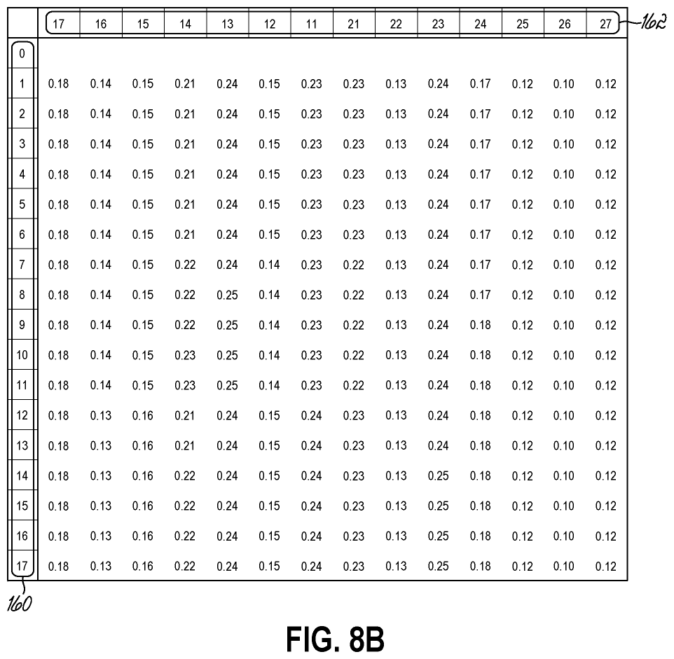

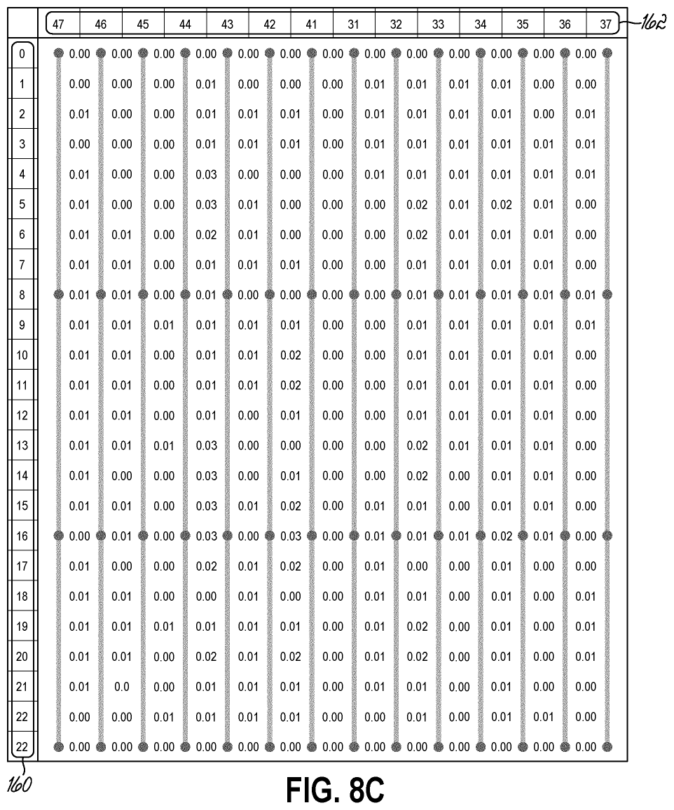

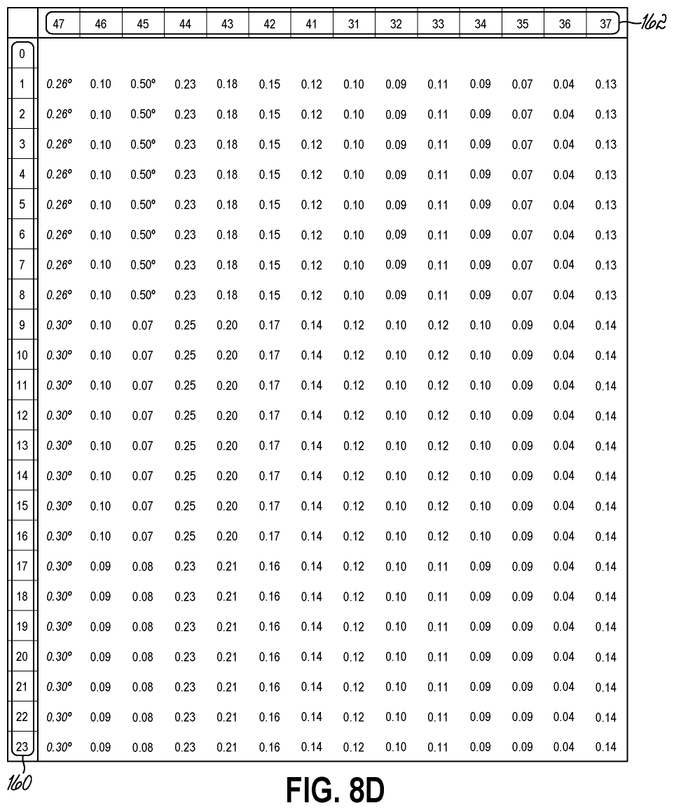

In an exemplary embodiment and with reference to FIG. 7, at 182, the staging solver algorithm 180 addresses a majority or all non-IPR contacts in FIGS. 6A and 6C, for example, non-IPR contacts 172, 174, and 176 shown in FIG. 6A for the exemplary T1 models 102 and 103 during orthodontic treatment planning. The resulting staging sequence is shown in FIGS. 8A and 8C, respectively, in which all non-IPR contacts, including specifically non-IPR contacts 172, 174, and 176 are resolved. FIGS. 8B and 8D tabulate the velocities associated with each stage and correspond to FIGS. 8A and 8C, respectively.

To resolve non-IPR contacts with the staging solver algorithm 180, at 186 in FIG. 7, the staging solver algorithm 180 creates at least one key frame in the sequence of staging shown in FIGS. 6A-6D. By way of example only, and not limitation, the modified staging chart is shown in FIG. 8A with a key frame 186 created at stage 6 of FIG. 6A. A key frame represents a tooth position and/or orientation that is modified from its initial staging position and/or orientation at one stage. Each key frame is described by two characteristics—a stage number and a tooth position. Because the tooth's position and/or orientation in a key frame is changed, the virtual movement pathway for that tooth deviates from its initial virtual movement pathway described with reference to FIG. 6A. For example, a virtual pathway may be constructed via linear interpolation between two positions of a model tooth, such as between the T1 position and the T2 position. A key frame between those positions changes the pathway so that it is no longer a linear interpolation between the two positions. More generally, the new pathway is no longer a linear interpolation of each degree of freedom of the model tooth's virtual motion from the T1 position to the T2 position.

With reference to FIG. 8A, a key frame may be created for each tooth at one stage. By way of example, a key frame 186 is one key frame in stage 6 and is indicated by a solid circle. As shown, solid circles are shown for each of stages 0, 6, 11, and 17 for tooth 17. Each circle is connected by a solid line, which indicates that tooth 17 is moving at each stage. The key frame 186 indicates that the position and/or orientation of the tooth 17 deviates at stage 6 (the key frame 186) from the initial pathway shown in FIG. 6A. The key frame 186 also changes the positions and/or orientations of tooth 17 in each of stages 1-5. From a different perspective, the position and/or orientation of the model tooth 11 at the key frame 186 modifies the positions and/or orientations of the model tooth 11 at each of stages 1-5. There is also a deviation in movement from the new, modified position and/or orientation at the key frame 186 through subsequent stages (in the example of FIG. 8A, stages 7-17 are modified relative to FIG. 6A). From a different perspective, the positions and/or orientations of the model tooth 17 at stages 7-17 depends on each of the positions and/or orientations of the model tooth 17 at the key frame 186 and the key frame (unlabeled) at stage 11. Thus, a key frame modifies a tooth's position and/or orientation at one stage and modifies two virtual movement pathways (to and from the key frame) for that tooth in view of its new position and/or orientation. As an additional example, by way of comparison of the staging shown in FIG. 6A to the staging shown in FIG. 8A, the staging solver algorithm 180 adds the key frame 190 at stage 11. The key frame 190 modifies the position and/or orientation of tooth 11 of the T1 model 102 (FIG. 3A) at the stage 11. Virtual movement of the tooth 11 is thus modified from its initial virtual movement pathway from stage 6 (also a key frame) to stage 11 (i.e., to key frame 190) and from stage 11 to stage 17 (i.e., from the key frame 190).

With reference to FIGS. 7 and 8A at 186, in one embodiment, the staging solver algorithm 180 adds at least one row of key frames at one stage of orthodontic treatment planning. That is, a key frame is added for each tooth position at the selected stage. This is referred to as a key-frame row. In the exemplary embodiments shown in FIGS. 8A and 8C, two key-frame rows (including key-frame 190 at tooth 11) are created at stages at ⅓ and ⅔ of the total number of stages. For example, one key-frame row 192 is at stage 6 and a second key-frame row 194 is at stage 11. The position and/or orientation of each tooth 11-17 and 21-27 is therefore modified from stage 1 to stage 6 per key-frame row 192 at stage 6. The position and/or orientation of each tooth 11-17 and 21-27 is therefore modified to and from stage 11 per key-frame row 192 and 194. Consequently, movement of each tooth 11-17 and 21-27 to and from stage 6 and to and from stage 11 are therefore modified. As is described below, the staging solver algorithm 180 optimizes each tooth position at stage 6 and at stage 11 and may also change the staging sequence of the stage number associated with one or both the key-frame rows 192 and 194. In other words, the staging solver algorithm 180 may change the key-frame row 192 from stage 6 to stage 5 or to stage 7. The key-frame row 194 may be alternatively or similarly modified. Changing the sequence of the key-frame rows 192, 194 is indicated by arrows 196 in FIG. 8A. It is contemplated that the key-frame rows 192, 194 may be moved by more than one stage in the sequence of stages shown in FIG. 8A.

In one embodiment and with reference to FIG. 7, the staging solver algorithm 180 resolves all non-IPR contacts. For example, the solver algorithm 180 resolves non-IPR contacts 172, 174, and 176 (and all others) shown in FIG. 6A. That is, tooth position and/or orientation is adjusted so that these contacts do not occur during movement. This is shown by way of comparison in FIG. 8A to FIG. 6A in which each of the non-IPR contacts (shown underlined in FIG. 6A) is resolved to a normal contact range. No non-IPR contacts are shown in FIG. 8A. In addition, advantageously, the staging solver algorithm 180 solves an issue of misaligned key-frames that are typically generated during manual manipulation of stages to rectify interproximal tooth collisions. In other words, during manual manipulation, key-frames may be added to different stages with a key frame being added to less than all teeth on a single stage. The staging solver algorithm 180 may eliminate individual key frames at individual ones of the teeth at multiple different stages. That is, the staging solver algorithm 180 creates a key frame for each tooth at a particular stage and so, in one embodiment, eliminates any necessity of creating a single key frame for a single tooth at another stage. Furthermore, one or the other or both the key-frame rows 192, 194 permit all key frames within a stage to be collectively moved with a single modification, as is indicated by arrow 196 in FIG. 8A. This simplifies the tooth movements for doctors and simplifies editing of the staging by clinicians.

More specifically, in one embodiment and with reference to FIGS. 7 and 8A, the staging solver algorithm 180 optimizes the position and/or the orientation of each tooth 11-17 and 21-27 of FIG. 3A in each key-frame row 192 and 194 and optimizes the staging sequence of the key-frame rows 192 and 194 in the overall sequence of stages. Although not described further herein, the staging solver algorithm 180 may additionally or alternatively optimize the position and/or the orientation of each tooth 31-37 and 41-48 of FIG. 3B in a similar manner.

In one embodiment, resolving non-IPR contacts and optimizing tooth position and/or orientation via the staging solver algorithm 180 at 182 in FIG. 7 includes using sequential quadratic programming methods. In that regard, the position and/or the orientation of each model tooth and staging sequence of a key-frame row are defined in a staging model. With the position and/or the orientation space between adjacent teeth are also defined. In one embodiment, tooth positions and spaces between adjacent teeth at each stage are each defined as non-linear problems. One or more inequality constraints to the non-linear problems define a linear inequality system. The non-linear problems are then solved as constrained by the linear inequality system. The non-linear problems are solved to cause a convergence of tooth positions at each stage in which the spacing between the teeth are preserved. This eliminates virtual tooth collision in the sequence of stages. Building the linear inequality system includes linearizing the non-linear problems by calculating partial derivatives of them. With coefficients derived from the partial derivative solutions substituted in the linear inequality system, the staging solver algorithm 180 solves the staging model as constrained by linear inequality system and according to a prioritization of the constraints defining the linear inequality system. The tooth position and/or orientation, spacing between teeth, and key-frame row staging sequence determined from one iteration of the solution may be used as a starting point for a subsequent iteration so that the tooth position and/or orientation, spacing between teeth, and key-frame row staging sequence converge to a solution which is constrained by the linear inequality system and so resolves all interproximal collisions at each stage. A description of sequential quadratic programming methods is found in “Fast Resolution of Hierarchized Inverse Kinematics with Inequality Constraints” by Adrien Escande et al. at https://hal.archives-ouvertes.fr/hal-00484853, submitted May 19, 2010, and is incorporated herein by reference in its entirety. As is indicated, the method disclosed reduces computation time relative to inverse kinematics with inequality constraints.

With reference to FIG. 7 at 200, in one embodiment, a staging model is defined. In the staging model, the i-th tooth position X at stage j in the virtual space is described as a vector with at least two components (α and β) transposed and written as:

[symbol]

=(α_{i,j},β_{i,j})^{T}i=1̅,̅N̅j=1̅,̅K̅ (1)

where N is the number of teeth, and K is related to the number of stages of treatment in the treatment plan. By way of example, K is greater than the number of stages of treatment.

A position of a key-frame row in the staging sequence of stage for orthodontic treatment is variable and is calculated according to vector:

\vec{T}=(Tₚ)^{T} (2)

where Tₚ is a float value position of the “p-th” intermediate key-frame row and p=1̅,̅ M̅ in which M is a number of key-frame rows between stage 0 (T1 model) and the last stage (T2 model). When in range [0, 1] this becomes:

0.0<T₁<T₂< . . . <T_{M}<1.0

As for K in (1) above, to determine interproximal collisions at each stage, embodiments of the invention control each tooth position and/or orientation in all stages and not in only in the key-frame row(s). The staging solver algorithm 180 determines collisions only between successive teeth in a jaw for all stages. To do so, the staging solver algorithm 180 discretizes the virtual movement pathway of each tooth from the T1 model 102, 103 to the T2 model 136, 137. Each virtual movement pathway is parametrized in range [0, 1] into K discrete stages. K must be large enough for accurate monitoring of collisions along each virtual movement pathway. By way of example and not limitation, K may range from 70 to 100 because treatment staging rarely, if ever, exceeds 100 stages. After finding a solution, unneeded stages may be removed. Thus, K may be ultimately related to the number of stages of treatment. This is the relation between K and the number of stages, that is, K is typically greater than the number of stages in a treatment plan. The staging sequence of a key-frame row is described and solved in the variable space with dimension=N*Q*M. As a summary, in the above equations,
1) α is buccal/lingual motion (translation in mm perpendicular to the arch form),
2) β is mesial/distal motion (translation in mm along the arch form),
3) N is the total number of teeth in the patient's arch,
4) Q is the degrees of freedom used to vary tooth position, and
5) M is the number of key-frame rows.

The goal of the staging solver algorithm 180 is to resolve interproximal collisions as identified in initial staging, for example, the staging solver algorithm 180 resolves interproximal collisions from the T1 model 102, 103 to the T2 model 136, 137 as is shown in FIGS. 6A and 6C. To construct the staging model, space Sᵢⱼ is defined between adjacent teeth i and i+1 at stage j. The space Sᵢⱼ is defined as a non-linear function Fⱼ( ) of teeth positions on each of p and p+1 key-frame rows where p is determined as Tₚ<Tⱼ<Tₚ₊₁ in which Tⱼ represents that for each stage j there exists two key-framed rows Tₚ and Tₚ₊₁ which determine teeth positions and collisions on the stage j in the staging sequence of key-frame rows Tₚ and Tₚ₊₁. In one embodiment, two key-frame rows Tₚ and Tₚ₊₁ are not consecutive stages. Instead, they are spaced apart by one or more stages. Space Sᵢⱼ may be positive or negative indicating interproximal space or adjacent tooth overlap, respectively. Collisions at any stage depend only on teeth position on two nearest key-frame rows to a given stage. For that reason, tooth position is varied only on key-frame rows. This determines teeth positions for all other stages and as a result addresses teeth collisions for these stages. For each stage j, the staging model includes different non-linear functions Fⱼ( ) By way of example, for each stage each non-linear function is of two variables, α (buccal/lingual motion) and β (mesial/distal motion). The non-linear functions Fⱼ( ) for different stages may then be different. However, because there are six degrees of freedom, there are other degrees of freedom that influence Sᵢⱼ. By way of example, these include tooth rotation, such as inclination and angulation.

Based on the above, the space Sᵢⱼ between adjacent teeth i and i+1 at stage j is represented by

Sᵢⱼ=Fⱼ(\vec{X_{ι,p}},\vec{X_{ι,p+1}},\vec{X_{ι+1,p}},\vec{X_{ι+1,p+1}},Tₚ,Tₚ₊₁) (3)

To avoid collisions between teeth on their respective virtual movement pathways, the space Sᵢⱼ is constrained according to the following inequality at each stage.

0<Sᵢⱼ<Sᵢ^{max}i=1̅,̅N̅−̅1̅j=1̅,̅K̅ (4)

where Sᵢ^{max} is the maximum space permitted between teeth i and i+1. The minimum space or lower limit of 0 is described for simplicity, but the lower limit may be changed in relation to the range of non-IPR contacts (e.g., 0.0 mm to 0.04 mm) described herein. The maximum space Sᵢ^{max} may be determined based on space between these teeth in both the T1 model 102, 103 and T2 model 136, 137.

In one embodiment, in addition to the linear inequality space constraint (4) above, the linear inequality system includes a linear inequality velocity constraint for tooth movement. This is a constraint that restricts the magnitude of tooth motion along its virtual movement pathway to less than a threshold value. Generally, the velocity constraint can be simplified for the two degrees of freedom for buccal/lingual motion a and mesial/distal motion β described above and written as:

|X_{i,j+1}^{q}−X_{i,j}^{q}|<Velocityₘₐₓ^{q},i=1̅,̅N̅j=1̅,̅K̅q=1̅,̅Q̅ (5)

where \vec{Velocityₘₐₓ}=(αₘₐₓ, βₘₐₓ)^{T} contains restrictions for each degree of freedom of tooth motion used. By way of example and not limitation, velocity constraints may be set to 0.25 mm translational movement (i.e., for each of α and β), 2 degrees rotational movement, and 1 degree of tip/torque movement per stage. Embodiments of the invention are not limited to Q=2 (degrees of freedom) as is described above with respect α and β. Accordingly, (5) may cover all degrees of freedom.

In one embodiment, an additional or alternative linear inequality constraint restricts the magnitude of total tooth motion (i.e., deviation) from its original position and/or orientation in the T1 model 102, 103. Thus, the linear inequality system may include a deviation constraint. Accordingly, each tooth is limited in how far it may be moved from its original position and/or orientation in the T1 model 102, 103. The deviation constraint is intended to keep the tooth in the patient's virtual jawbone. A simplified expression written for both buccal/lingual motion α and mesial/distal motion β is:

|X_{i,j}^{q}−X_{i,0}^{q}|<Deviationₘₐₓ^{q},i=1̅,̅N̅j=1̅,̅K̅q=1̅,̅Q̅ (6)

where \vec{X_{ι,0}}=(α_{i,0}, β_{i,0})^{T} is tooth position at T1.

In one embodiment, with reference to FIG. 7, at 202, there are multiple solution goals for the linear inequality system. With reference to FIG. 9, these goals are prioritized prior to solving the staging model. That is, in the exemplary embodiment, the solution goals to linear inequality constraints (4), (5), and (6) are prioritized. The established priority ensures that the orthodontic treatment plan developed will be a realistic, patient-centric, plan. While a particular priority to the goals is described herein, the goals may be prioritized in a different order. Furthermore, it will be appreciated that additional and/or alternative linear inequality constraints may be possible. As such, embodiments of the invention are not limited to the specific priority disclosed unless stated otherwise.

Accordingly, with reference to FIGS. 7 and 9, the staging solver algorithm 182 assigns the goal of resolving interproximal collisions, that is, maintaining Sᵢⱼ in the indicated range according to (4), as the highest priority task (i.e., priority number 1). The patient's teeth should not collide during orthodontic treatment. This is the primary goal to achieve efficient orthodontic treatment. For example, the goal of solving for Sᵢⱼ should not include a collision. To avoid that possibility, staging should indicate no collisions (e.g., normal contacts between 0 mm and 0.04 mm) between any two teeth at any stage.

In accordance with FIG. 9, in one embodiment, the deviation constraint (6) is assigned a priority after Sᵢⱼ (i.e., in this embodiment, priority number 2). Thus the total magnitude of tooth movement should be limited. The next priority goal is the velocity constraint (5), and it is assigned priority number 3.

In one embodiment and with reference to FIG. 7 at 204 and following prioritization of the goals of the linear inequality system, the staging solver algorithm 180 linearizes the staging model, which is the math model described above including the linear inequality system, priorities, iterated with linearization using partial derivatives. This may include initial linearization of Fⱼ( ) near some initial solution. This may include some initial estimation for teeth movement from the T1 model to the T2 model. For example, this could be the virtual pathway illustrated in FIGS. 6A and 6C in which linear interpolation is used to determine stages between a start location (T1) and an end location (T2).

To that end, in one embodiment, (2) above may be rewritten with a single vector as follows,

\vec{V_{ι,p}}=(\vec{X_{ι,p}},\vec{X_{ι,p+1}},\vec{X_{ι+1,p}},\vec{X_{ι+1,p+1}},Tₚ,Tₚ₊₁) (7)

With the single vector \vec{V_{ι,p}}, the staging solver algorithm 180 linearizes Sᵢⱼ ((3) above) by determining a partial derivative at each iteration as:

$${s}_{i , j}^{i t e τ + 1} = {s}_{i , j}^{i t e τ} + {( \frac{\partial {F}_{j}}{\partial \vec{{V}_{l , p}}} )}^{iter} * \vec{{v}_{l , p}}$$

where S_{i,j}^{iter} is the spacing Sᵢⱼ for a particular iteration (“iter”). The linear inequality spacing constraint (4) with variables \vec{V_{i,p}} is written as:

$$0 - {S}_{i , j}^{i t e r} < {( \frac{\partial {F}_{j}}{\partial \vec{{V}_{i , p}}} )}^{i t e r} * \vec{{v}_{i , p}} < {S}_{i}^{max} - {S}_{i , j}^{iter} i = \overline{1 , N} j = \overline{1 , K} \qquad ( 8 )$$

Velocity (5) and Deviation (6) may be used as simple inequalities without linearization. In one embodiment, all partial derivatives of (5) and (6) equal 1. Thus, the staging model including each of the spacing (in terms of \vec{V_{ι,p}}), velocity, and deviation constraints are linearized.

In one embodiment and with reference to FIG. 7, at 204, the staging solver algorithm 180 computes partial derivatives of mesial-distal and buccal-lingual movements for each tooth of each keyframe row 192 and 194. For example, the staging solver algorithm 180 computes partial derivatives of spaces with respect to tooth positions \vec{X_{ι,j}} in each of mesial-distal and buccal-lingual movements.

At 206 in FIG. 7, the staging solver algorithm 180 computes the partial derivatives of spaces with respect to time movement of key-frame rows 192 and 194. This may be achieved by solving (7), which contains positions of key-framed rows Tₚ, Tₚ₊₁, by calculating partial derivatives of collisions

$$\frac{\partial {F}_{j}}{\partial \vec{{V}_{i , p}}}$$

by all components of the vector including positions of key-framed rows Tₚ, Tₚ₊₁. It is noted that calculating partial derivatives on discrete values of key-framed rows 192 and 194 may be achieved by representing a stage of the key-framed rows as continuous variables, e.g.,

0.0≤T₁<T₂< . . . <T_{M}<1.0

from above. After solution to \vec{T} (according to (2)) is determined, it is discretized on a predetermined number of fixed discrete stages, for example 100. Based on velocity, the number of stages is adjusted (e.g., decreased) to the number of stages required for treatment.

At 210, partial derivatives from 204 and 206 are substituted into the linear inequality system, and the system is solved for each tooth in the key-frame row. In one embodiment, teeth collisions (i.e., spacing for iteration, s_{i,j}^{iter}) is separately calculated from velocity (i.e., |X_{i,j+1}^{q}−X_{i,j}^{q}|) for the key-famed rows. It is contemplated that this is possible because key-framed rows include all teeth in the respective jaw. After resolving collisions, velocity violations can be resolved at one step with the determined increment in stage number. In one embodiment, if the velocity constraint (5) remains too high or too low, the staging solver algorithm 180 increases the number of stages or decreases the number of stages, respectively, by a predetermined number (e.g., 3 to 7 stages), and then solves each of (4)-(6) until all each solution satisfies the constraints. Further, in one embodiment, not only does the staging solver algorithm 180 vary the sequence of one or more of the key-frame rows but the staging solver algorithm 180 also varies the final stage corresponding to the T2 model stage, T_{T2}. In this way, velocity constraints can be satisfied by adding or removing stages to increase or reduce treatment length, respectively, as determined by the staging solver algorithm 180.

Once the staging model is solved at 210, the staging solver algorithm 180 applies the determined geometrical and time movement solutions to the key-frame rows 192 and 194. If collisions remain between any adjacent teeth at their respective positions X_{i,j}, that is, Sᵢⱼ>Sᵢ^{max}, then the staging solver algorithm 180 iterates the calculation at 214 until Sᵢⱼ converges in the constrained range according to (4). As shown, during iteration at 214, partial derivatives for movement are calculated at the new tooth position X_{i,j} to provide a new Sᵢⱼ, and so on until Sᵢⱼ>Sᵢ^{max}. This may be a similar process as in the Newton-Raphson method. During iterations, if there are tooth collisions, the staging solver algorithm 180 modifies the tooth position X_{i,j} to a more ideal tooth position to avoid that collision. This may initially produce an increase is the magnitude of tooth collisions, i.e., more negative Sᵢⱼ. Once the staging model is converged, the stages for orthodontic treatment are displayed as shown by way of example in FIGS. 8A-8D.

In one embodiment, to solve the linear inequalities system additional technical constraints may be required. These may also be prioritized at 202. With reference to FIG. 9, these may include, by way of example only, (1) Tₚ<Tₚ₊₁ p=1̅,̅ M̅ to have key-framed rows sorted in increasing order. This may be set as the highest priority of the technical constraints. As a second technical constraint (2), a \vec{v_{ι,p}} incremental step is restricted on each iteration so that linearization is applicable. This may be set as the lowest priority. A third technical constraint (3) is \vec{v_{ι,p}}=\vec{0} to avoid ambiguity in case of prioritized linear inequalities system is underdefined, lowest priority. The staging solver algorithm 180 can resolve collisions in any number of ways. Among that plurality of solutions, the most optimum is with minimal teeth motion. The third technical constraint is designed to achieve minimal teeth motion during convergence.

Once the staging model is converged on a solution, in one embodiment and with reference to FIG. 5 at 184, orthodontic treatment planning 142 includes consideration of any difference in the number of stages between the patient's upper arch and the patient's lower arch. If the difference is less than 5 aligners, stages and aligners may be added to the arch with the fewest stages. In essence, this stretches the number of stages on the arch with less aligners to match the one with the most aligners. If the difference is more than 5, passive aligners may be created by adding stages after the last calculated treatment stage. The added stages may not move the teeth but may provide some comfort value to the patient as treatment continues on the patient's other jaw.

Following proper orthodontic treatment planning, information concerning the stages may be utilized to manufacture aligners corresponding to each stage at 144 in FIG. 5. Although not shown, an exemplary process of forming the aligner 126 may include deforming a workpiece with a mold. The mold may be manufactured according to a corresponding computer model of the patient's dentition at a stage of the orthodontic treatment plan. For example, a 3-D digital model of the patient's teeth corresponding to a stage of the treatment plan may be utilized to produce a mold. For example, a series of computer models corresponding to the stages of FIGS. 8A and 8C provides incremental steps from the T1 model to one or more intermediate dentition models and ending with the T2 model are generated and may be used to produce corresponding molds. An exemplary method of forming the molds and manufacturing aligners is described in commonly owned U.S. Pat. No. 10,935,958, which is incorporated by reference herein in its entirety.

With reference to FIG. 10, as a result, the aligner 126 includes a hollow shell 250 that is configured to encapsulate one or more crowns of a patient's teeth. The shell 250 is formed with a plurality of cavities 252 that collectively define an edge 254, which defines an opening 256. Each cavity 252 is shaped to receive a specific one of the patient's teeth through the opening 256 during use of the aligner 126. The shell 250 is made of an elastic material in one or more layers and may include one or more receptacles 258 that are configured to receive an attachment (not shown) on the patient's tooth and/or one or more devices in the aligner 126.

During orthodontic treatment, the aligner 126 is selectively positioned over the patient's teeth and may fit tightly due to slight differences in the position of one or more of the cavities 252 relative to the corresponding tooth. A forcible contact with the aligner 126 may move the patient's teeth toward a predetermined position according to a patient's treatment plan that may ultimately end at T2. A set of aligners (not shown) may include one or more aligners 126. During orthodontic treatment, each stage of treatment may include an aligner that provides slightly different movement of the patient's teeth. The individual aligners are utilized in a predetermined sequence to complete orthodontic treatment or move the patient's teeth to T2. Accordingly, each aligner in the series may move one or more teeth a prescribed amount. While similar, each aligner is slightly different in shape. Cumulatively, these individual amounts may result in complete treatment of the patient's malocclusion.

As is described above, according to embodiments of the invention, a practitioner implements the computer 106 (e.g., shown in FIGS. 2 and 11) with software capable of generating and manipulating the 3-D digital models in accordance with the system 104 described above. Referring now to FIG. 11, embodiments of the invention described above, or portions thereof, such as the system 104 may be implemented using one or more computer devices or systems, such as exemplary computer 106. The computer 106 may include a processor 302, a memory 304, an input/output (I/O) interface 306, and a Human Machine Interface (HMI) 308, such as a display 308 of FIG. 2. The computer 300 may also be operatively coupled to one or more external resources 310, such as an intra-oral scanner and CBCT system or manufacturing system 124, via a network 122 and/or I/O interface 306. External resources may include, but are not limited to, servers, databases, mass storage devices, peripheral devices, cloud-based network services, or any other resource that may be used by the computer 106.

The processor 302 may include one or more devices selected from microprocessors, micro-controllers, digital signal processors, microcomputers, central processing units, field programmable gate arrays, programmable logic devices, solid state machines, logic circuits, analog circuits, digital circuits, or any other devices that manipulate signals (analog or digital) based on operational instructions that are stored in memory 304. Memory 304 may include a single memory device or a plurality of memory devices including, but not limited to, read-only memory (ROM), random access memory (RAM), volatile memory, non-volatile memory, static random access memory (SRAM), dynamic random access memory (DRAM), flash memory, cache memory, and/or data storage devices such as a hard drive, optical drive, tape drive, volatile or non-volatile solid state device, or any other device capable of storing data.

The processor 302 may operate under the control of an operating system 314 that resides in memory 304. The operating system 314 may manage computer resources so that computer program code embodied as one or more computer software applications or algorithms, such as an application 316 including the staging solver algorithm 180 residing in memory 304, may have instructions executed by the processor 302. In an alternative embodiment, the processor 302 may execute the application 316 directly, in which case the operating system 314 may be omitted. One or more data structures 318, for example the 3-D digital models 102, 103, 136, and 137, may also reside in memory 304, and may be accessed and modified by the processor 302, operating system 314, or application 316 (e.g., according to the staging solver algorithm 180) and may be manipulated by the clinician.

The I/O interface 306 may provide a machine interface that operatively couples the processor 302 to other devices and systems, such as the external resource 310 or the network 122. The application 316 may thereby work cooperatively with the external resource 310 or network 122 by communicating via the I/O interface 306 to provide the various features, functions, applications, processes, or modules comprising embodiments of the invention. The application 316 may also have program code that is executed by one or more external resources 310, or otherwise rely on functions or signals provided by other system or network components external to the computer 106. Indeed, given the nearly endless hardware and software configurations possible, persons having ordinary skill in the art will understand that embodiments of the invention may include applications that are located externally to the computer 106, distributed among multiple computers or other external resources 310, or provided by computing resources (hardware and software) that are provided as a service over the network 312, such as a cloud computing service.

The HMI 308 may be operatively coupled to the processor 302 of computer 106 in a known manner to allow a practitioner to interact directly with the computer 106 to, for example, operate user interface. The HMI 308 may include video or alphanumeric displays, a touch screen, a speaker, and any other suitable audio and visual indicators capable of providing data to the user. The HMI 308 may also include input devices and controls such as an alphanumeric keyboard, a pointing device, keypads, pushbuttons, control knobs, microphones, etc., capable of accepting commands or input from the user and transmitting the entered input to the processor 302.

A database 320 may reside in memory 304 and may be used to collect and organize data used by the various systems and modules described herein. The database 320 may include data and supporting data structures, for example 3-D digital model 102, 103, 136, 137, that store and organize the data. In particular, the database 320 may be arranged with any database organization or structure including, but not limited to, a relational database, a hierarchical database, a network database, or combinations thereof. A database management system in the form of a computer software application executing as instructions on the processor 302 may be used to access the information or data stored in records of the database 320 in response to a query, where a query may be dynamically determined and executed by the operating system 314, other applications 316, or one or more modules.

In general, the routines executed to implement the embodiments of the invention, whether implemented as part of an operating system or a specific application, component, program, object, module or sequence of instructions, or a subset thereof, may be referred to herein as “computer program code,” or simply “program code.” Program code typically comprises computer-readable instructions that are resident at various times in various memory and storage devices in a computer and that, when read and executed by one or more processors in a computer, cause that computer to perform the operations necessary to execute operations and/or elements embodying the various aspects of the embodiments of the invention. Computer-readable program instructions for carrying out operations of the embodiments of the invention may be, for example, assembly language or either source code or object code written in any combination of one or more programming languages.

Various program code described herein may be identified based upon the application within which it is implemented in specific embodiments of the invention. However, it should be appreciated that any particular program nomenclature which follows is used merely for convenience, and thus the invention should not be limited to use solely in any specific application identified and/or implied by such nomenclature. Furthermore, given the generally endless number of manners in which computer programs may be organized into routines, procedures, methods, modules, objects, and the like, as well as the various manners in which program functionality may be allocated among various software layers that are resident within a typical computer (e.g., operating systems, libraries, API's, applications, applets, etc.), it should be appreciated that the embodiments of the invention are not limited to the specific organization and allocation of program functionality described herein.

The program code embodied in any of the applications/modules described herein is capable of being individually or collectively distributed as a program product in a variety of different forms. In particular, the program code may be distributed using a computer-readable storage medium having computer-readable program instructions thereon for causing a processor to carry out aspects of the embodiments of the invention.

Computer-readable storage media, which is inherently non-transitory, may include volatile and non-volatile, and removable and non-removable tangible media implemented in any method or technology for storage of data, such as computer-readable instructions, data structures (e.g., the 3-D digital model 102, 103, 136, and 137), program modules, or other data. Computer-readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired data and which can be read by a computer. A computer-readable storage medium should not be construed as transitory signals per se (e.g., radio waves or other propagating electromagnetic waves, electromagnetic waves propagating through a transmission media such as a waveguide, or electrical signals transmitted through a wire). Computer-readable program instructions may be downloaded to a computer, another type of programmable data processing apparatus, or another device from a computer-readable storage medium or to an external computer or external storage device via a network.

Computer-readable program instructions stored in a computer-readable medium may be used to direct a computer, other types of programmable data processing apparatuses, or other devices to function in a particular manner, such that the instructions stored in the computer-readable medium produce an orthodontic appliance including instructions that implement the functions, acts, and/or operations specified in the flow-chart, sequence diagram, and/or block diagrams. The computer program instructions may be provided to one or more processors of a general purpose computer, a special purpose computer, or other programmable data processing apparatus to produce a machine, such that the instructions, which execute via the one or more processors, cause a series of computations to be performed to implement the functions, acts, and/or operations specified in the flow-charts, sequence diagrams, and/or block diagrams.

### EXAMPLE

With reference to FIGS. 3A, 3B, 4A, 4B, 6A-6D, and 8A-8D, results for an exemplary calculation are shown for Q=2 (for two degrees of freedom), M=2 (the number of key-frame rows is 2), N=14 (the number of teeth in the arch is fourteen). Calculations in accordance with the staging solver algorithm above were conducted on an initial staging plan for the T1 model shown in FIG. 3A and FIG. 3B. An initial staging plan for the T1 model of FIG. 3A is shown in FIGS. 6A and 6B. An initial staging plan for the T1 model shown in FIG. 3B is shown in FIGS. 6C and 6D. Each initial staging plan of FIGS. 6A-6D was calculated with each degree of freedom of each tooth's virtual motion being linearly interpolated from stage 0 to a last stage, e.g., stage 17 in FIGS. 6A and 6B, and e.g., stage 22 in FIGS. 6C and 6D. Note that both the initial staging plans shown in FIGS. 6A and 6C each includes tooth-to-tooth collisions (referred to as non-IPR contacts). The collisions are indicated by underlining of values indicating a negative space between adjacent teeth. As shown, the collisions predominately occur between teeth 16 and 15, 13 and 12, and 12 and 11 starting at stage 3 and continuing through stage 16 of FIG. 6A. The maximum collision occurs between tooth 13 and 12 at stage 9 at which 0.10 mm of virtual overlap is observed. In FIG. 6C, the collisions occur between teeth 47 and 46, 46 and 45, 44 and 43, 43 and 42, 31 and 32, and 33 and 34. A maximum collision of 0.13 mm occurs between tooth 42 and 41 at stages 9-11.

Calculating the space Sᵢⱼ and velocity |X_{i,j+1}^{q}−X_{i,j}^{q}| in accordance with the staging solver algorithm provides the staging plans shown in FIGS. 8A-8D, in which FIGS. 8A and 8B correspond to staging plans for treatment of the T1 model shown in FIG. 3A to the T2 model shown in FIG. 4A, and in which FIGS. 8C and 8D correspond to staging plans for treatment of the T1 model shown in FIG. 3B to the T2 model shown in FIG. 4B. As shown, key-frame rows occur at stages 6 and 11 in FIG. 8A and at stages 8 and 16 in FIG. 8C. All collisions of FIGS. 6A and 6C are addressed. That is, no collisions exist in either FIGS. 8A and 8C. With reference to FIGS. 8B and 8D, velocities are in range.

While the present invention has been illustrated by a description of various embodiments and while these embodiments have been described in some detail, it is not the intention of the inventors to restrict or in any way limit the scope of the appended claims to such detail. Thus, additional advantages and modifications will readily appear to those of ordinary skill in the art. The various features of the invention may be used alone or in any combination depending on the needs and preferences of the user.

What is claimed is:

1. A computer-implemented method for orthodontic treatment planning with one or more aligners, the method comprising:
determining an initial virtual pathway for each model tooth in the patient's jaw that is to be moved during orthodontic treatment, each initial virtual pathway being from a first position and/or orientation of a model tooth in a first arrangement to a second position and/or orientation of the model tooth in a second arrangement, wherein each initial virtual pathway is divided into a plurality of stages with each stage of the plurality of stages defining a position and/or orientation of a respective one model tooth on the initial virtual pathway;
calculating a space between adjacent pairs of model teeth at each stage;
defining a minimum space between adjacent pairs of model teeth permitted for orthodontic treatment; and
when one or more of the calculated spaces is less than the minimum space, creating at least one key-frame row in which each tooth position and/or orientation in the stage is a key frame, and while one or more of the calculated spaces is less than the minimum space, iterating one or more times of:
for each model tooth at the at least one key-frame row, varying the position and/or orientation of the model tooth from the first position and/or orientation or from a previous position and/or orientation from a previous iteration; and
calculating a space between adjacent pairs of model teeth at each stage with the varied position and/or orientation of each model tooth at the at least one key-frame row,
wherein after iterating, the plurality of stages, in which each calculated space is greater than the minimum space, defines an orthodontic treatment plan in which each stage corresponds to an aligner for orthodontic treatment of a patient.

2. The method of claim 1, further comprising:
manufacturing a series of aligners for the patient with each aligner corresponding to one stage of the plurality of stages.

3. The method of claim 1, wherein iterating includes varying a position of the at least one key-frame row in the plurality of stages.

4. The method of claim 3, wherein varying the position includes moving the at least one key-frame row by one stage earlier or one stage later in the plurality of stages.

5. The method of claim 1, wherein the at least one key-frame row includes a first key-frame row and a second key-frame row and wherein iterating one or more times includes varying the position and/or orientation of each model tooth at each of the first key-frame row and the second key-frame row.

6. The method of claim 5, wherein the first key-frame row and the second key-frame row are not consecutive stages.

7. The method of claim 1, wherein the minimum space is in a range of 0.0 mm to 0.04 mm.

8. The method of claim 1, further comprising:
defining a maximum space, and wherein iterating occurs while one or more calculated spaces is less than the minimum space or is more than the maximum space.

9. The method of claim 1, wherein the position and/or orientation of each model tooth at each stage of the plurality of stages is defined in at least two degrees of freedom.

10. The method of claim 1, wherein the position and/or orientation of each model tooth is described as a vector with at least two components.

11. The method of claim 10, wherein the vector is: [symbol]
=(α_{i,j},β_{i,j})^{T} (1)
where X_{i,j} is the position and/or orientation of model tooth i at stage j in which i=1̅,̅ N̅ where N is a total number of model teeth in the patient's arch, j=1̅,̅ K̅ where K is related to a number of stages j of the plurality of stages, α is buccal/lingual motion of tooth i at stage j, and β is mesial/distal motion of tooth i at stage j.

12. The method of claim 11, wherein the at least one key-frame row includes a first key-frame row (Tₚ) and a second key-frame row (Tₚ₊₁) and the calculated space (Sᵢⱼ) between adjacent pairs of model teeth (i and i+1) at stage j is represented by:
Sᵢⱼ=Fⱼ(\vec{X_{ι,p}},\vec{X_{ι,p+1}},\vec{X_{ι+1,p}},\vec{X_{ι+1,p+1}},Tₚ,Tₚ₊₁) (3)
in which calculated space (Sᵢⱼ) is a function Fⱼ at stage j of \vec{X_{ι,p}}, which is the position and/or orientation of model tooth i at the first key-frame row, \vec{X_{ι,p+1}}, which is the position and/or orientation of model tooth i at the second key-frame row, \vec{X_{ι+1,p}}, which is the position and/or orientation of model tooth i+1 at the first key-frame row, and \vec{X_{ι+1,p+1}}, which is the position and/or orientation of model tooth i+1 at the second key-frame row.

13. The method of claim 12, wherein stage j is between the first key-frame row (Tₚ) and the second key-frame row (Tₚ₊₁).

14. The method of claim 12, wherein defining the minimum space as zero and further including defining a maximum space between adjacent pairs of model teeth according to an inequality:
0<Sᵢⱼ<Sᵢ^{max} (4)
where Sᵢ^{max} is the maximum space between adjacent teeth i and i+1.

15. The method of claim 1, wherein the method further comprises:
defining a maximum movement velocity of each model tooth between any two consecutive stages and while one or more of the calculated spaces is less than the minimum space, calculating a movement velocity between each consecutive stage for each model tooth, and
when the calculated movement velocity is greater than the maximum movement velocity, for each model tooth at the at least one key-frame row, varying the position and/or orientation of the model tooth from the first position and/or orientation or from a previous position and/or orientation from a previous iteration.

16. The method of claim 1, wherein the method further comprises:
defining a deviation constraint for total movement of each model tooth from the first position and/or orientation;
while one or more of the calculated spaces is less than the minimum space, calculating the total movement of each model tooth from the respective first position and/or orientation; and
when the calculated total movement is greater than the deviation constraint, for each model tooth at the at least one key-frame row, varying the position and/or orientation of the model tooth from the first position and/or orientation or from a previous position and/or orientation from a previous iteration.

17. A computer-implemented method for orthodontic treatment planning with one or more aligners in which an initial staging plan includes a plurality of stages of treatment and indicates one or more collisions between adjacent pairs of model teeth, the method comprising:
defining a minimum space between adjacent pairs of model teeth;
creating at least one key-frame row in the initial staging plan, wherein in the at least one key-frame row, each tooth position and/or orientation is a key frame; and
while at least one collision exists in the staging plan, iterating one or more times of:
for each model tooth at the at least one key-frame row, varying the position and/or orientation of the model tooth from an initial position and/or orientation or from a previous position and/or orientation from a previous iteration; and
calculating a space between adjacent pairs of model teeth at each stage with the varied position and/or orientation of each model tooth at the at least one key-frame row,
wherein after iterating, the plurality of stages, in which each calculated space is greater than the minimum space, defines an orthodontic treatment plan in which each stage corresponds to an aligner for orthodontic treatment of a patient.

18. The method of claim 17, further comprising:
manufacturing a series of aligners for the patient with each aligner corresponding to one stage of the plurality of stages.

19. The method of claim 17, wherein iterating includes moving the at least one key-frame row by one stage earlier or one stage later from a position in the initial staging plan.

20. The method of claim 17, wherein the at least one key-frame row includes a first key-frame row and a second key-frame row and wherein iterating one or more times includes varying the position and/or orientation of each model tooth at each of the first key-frame row and the second key-frame row.

21. The method of claim 20, wherein the first key-frame row and the second key-frame row are not consecutive stages.

22. The method of claim 17, wherein the minimum space is in a range of 0.0 mm to 0.04 mm.

23. The method of claim 17, further comprising:
defining a maximum space, and wherein iterating occurs while one or more calculated spaces is less than the minimum space or is more than the maximum space.

24. The method of claim 17, wherein the position and/or orientation of each model tooth is described as a vector with at least two components.
